(19) United States
(12) Patent Application Publication
(10) Pub. No.: US 2008/0274202 A1
(43) Pub. Date: Nov. 6, 2008

(54) Compositions and Method for Brain Specific Targeted Delivery of Therapeutic Agents

(76) Inventors: Richard P. Kraig, Chicago, IL (US); Michael Kaminski, Lockport, IL (US)

Correspondence Address: MICHAEL BEST & FRIEDRICH LLP, ONE SOUTH PINCKNEY STREET, P O BOX 1806, MADISON, WI, 53701, US

(21) Appl. No.: 11/997,476

(22) PCT Filed: Aug. 1, 2006
(86) PCT No.: PCT/US2006/029774

§ 371 Date: Apr. 18, 2008

Related U.S. Application Data
(60) Provisional application No. 60/704,639, filed on Aug. 1, 2005.
(60) Provisional application No. 60/723,189, filed on Oct. 3, 2005.

(51) Int. Cl. A61K 9/51; A61K 35/12; A61K 39/395; A61K 31/7088; A61P 25/00

(52) U.S. Cl. 424/491; 424/93.71; 424/490; 424/497

(57) ABSTRACT

Disclosed are methods and compositions for delivering a therapeutic agent to target organs or tissues, such as brain. The methods and compositions use bone marrow stem cells, monocytes, macrophages or microglial cells to deliver the therapeutic agent associated with nanoparticles to the target organ or tissue.

### CROSS REFERENCE TO RELATED APPLICATIONS

This application claims priority to U.S. Provisional Application Nos. 60/704,639, filed Aug. 1, 2005, and 60/723,189 filed Oct. 3, 2005, the subject matter of each of which is hereby fully incorporated by reference.

### INTRODUCTION

Targeted delivery of therapeutic agents to brain tissue or specific cells within the brain has immense potential for treatment of neurological disease. The structure and composition of the braih, however, presents certain challenges to delivery of therapeutics. The brain consists of five vastly different cell types (i.e., neurons, astrocytes, oligodendrocytes, microglia and endothelial cells) which together form the neurovascular unit. Expression of a targeted polynucleotide may vary between each cell type, depending on local stimuli, triggering variable net tissue effects. Also, the “blood-brain barrier” (BBB) serves as a selectively permissive impediment to entry and exit of cells and molecules. Therefore, ideally, targeted delivery should employ a vehicle that permits passage across the blood-brain barrier and which targets the specific cell type of interest.

Because macrophages and microglia are typically difficult to transfect, replication-deficient viral vectors have become most popular for delivery to such cells. However, viral vectors are undesirable in that they can induce inflammatory reactions and carry the potential for reversion to virulent form.

Targeting gene-modified hematopoietic cells to brain has been explored by irradiating the body to first kill all endogenous dividing cells, and transferring genetically altered monocytes and macrophages to the organisms. Newly tagged monocytes, macrophages and microglia migrate into nervous tissue, where differentiation to microglia occurs. However, whole body irradiation carries with it substantive side effects, including cognitive decline from brain exposure to radiation. Immortalized microglia transfected in vitro may be delivered to the brain following arterial injection, but delivery of immortalized cells carry inherent risks. The present invention is not associated with the confounding factors noted above.

### BRIEF DESCRIPTION OF THE DRAWINGS

FIG. 1 is a drawing showing the composition of an mPEG-PLGA nanoparticle.

FIG. 2 is a drawing showing the induction of spreading depression in the brain of rat, and neocortical and hippocampal c-fos staining.

FIG. 3 A, B and C are photographs showing the structure of various nanoparticles. D and E are graphs showing the size distribution of nanoparticles and the amount of DNA released from nanoparticles at various time points.

FIG. 4 is a photograph showing the integrity of DNA released from nanospheres.

FIG. 5 A, B and C are photographs of electron micrographs of microglial cells, comprising (B and C) or not comprising (A) nanoparticles. D is a photograph of a confocal image of fluorescing microglia comprising coumarin-loaded nanoparticles. E is a photograph of the microglia of D taken using transmitted light.

FIG. 6 A, B, C and D are photographs showing bright field images of GFP protein expression in microglia. E and F are photographs showing confocal images of GFP protein expression in microglia.

FIGS. 7 A and B are photographs showing fluorescence microscopic images of microglia comprising GFP pseudo-colored with “fire” LUT (A) and Nomarski images of the same cells (B).

FIG. 8 A, B and C are photographs showing confocal fluorescence intensity of microglia comprising coumarin-containing nanoparticles. D and E are photographs showing confocal fluorescence intensity of HOTC cells incubated with microglia comprising coumarin-loaded nanoparticles (D), or coumarin-loaded nanoparticles (E).

FIGS. 9 A and B are photographs showing confocal fluorescence intensity of HOTC cells exposed to microglia containing coumarin-loaded nanoparticles and having undergone spreading depression (A) or not (B).

### SUMMARY OF THE INVENTION

In one aspect, the present invention provides a method of delivering a therapeutic agent to the brain of a mammal. The method involves contacting a bone marrow stem cell, monocyte, macrophage, or microglia ex vivo with a nanoparticle comprising a therapeutic agent under conditions that allow endocytosis of the nanoparticle by the cell. The cell is then delivered to the brain.

In another aspect, the present invention provides a method of producing a bone marrow stem cell, monocyte, macrophage or microglial cell for delivering a therapeutic agent to a tissue. The bone marrow stem cell, monocyte, macrophage or microglial cell is contacted ex vivo with a nanoparticle comprising a therapeutic agent, under conditions that allow endocytosis of the nanoparticle by the cell. The therapeutic agent includes a polynucleotide.

The present invention further provides a composition for delivering a therapeutic agent to a tissue. The composition includes a bone marrow stem cell, monocyte, macrophage or microglial cell comprising a nanoparticle comprising the therapeutic agent, and a medium. The medium is substantially free of serum or protein.

In another aspect, the present invention provides a composition for delivering a therapeutic agent to a tissue. The composition includes a bone marrow stem cell, monocyte, macrophage or microglial cell comprising a nanoparticle comprising the therapeutic agent. The therapeutic agent includes a polynucleotide.

### DETAILED DESCRIPTION

In one embodiment, the present invention is directed to loading nanoparticles carrying therapeutic agents into microglia, monocytes, macrophages and/or related bone marrow stem cells ex vivo for subsequent delivery into whole animals, for example, mammals. Delivery may be suitably accomplished via direct injection into brain or via traversal of the blood-brain barrier, such as by administration to another part of the body (e.g., intra-arterial, intra-thecal, intra-peritoneal, or intra-nasal administration).

Microglia are resident immune cells of the brain derived from bone marrow stem cells. During development, certain bone marrow cells enter the blood and differentiate into monocytes. Monocytes in turn differentiate into macrophages in tissues. Macrophages further differentiate into organ-specific immune cells, such as microglia in brain. The bone marrow stem cells referred to herein are those bone marrow stem cells that are precursors of a microglia or have the capacity to differentiate into a microglia.

Microglia, macrophages, monocytes and bone marrow stem cells (MMMsc) have a capacity to engulf small objects such as nanoparticles. Microglia and macrophages are found in adult brain under normal circumstances and may concentrate to areas of disease. Microglia are motile within the brain, distribute evenly when added to the tissue and are highly sensitive to changes in both brain and immune function. Suitably, microglia, macrophages, monocytes and bone marrow stem cells (MMMsc) may be loaded ex vivo with nanoparticles carrying therapeutic agents, and delivered to brain or to target regions within the brain, wherein the nanoparticle or therapeutic agent may be released and taken up by cells of the brain or target region of the brain.

As used herein a “nanoparticle” is a particle of submicron dimensions. Optionally, the nanoparticle is comprised of polymeric materials. Suitably, the nanoparticle may be comprised of natural or synthetic polymeric materials. As used herein, “synthetic polymeric materials” do not include natural polymers, such as proteins or starch. Examples of suitable polymeric materials include, but are not limited to homopolymers, copolymers, random polymers, graft polymers, alternating polymers, block polymers, branch polymers, arborescent polymers and dendritic polymers. Nanoparticles include nanospheres, which are nanoparticles having a substantially round, spherical or globular structure. Nanoparticles of the present invention may be used to carry therapeutic agents for delivery to target cells or tissue. As used herein, carrying of a therapeutic agent by a nanoparticle includes encapsulation of the therapeutic agent by the nanoparticle, or attachment, adsorbtion or other association of the therapeutic agent to or with the nanoparticle. Suitably, nanoparticles may be biodegradable, for example being made of FDA-approved polymers and reagents for internal use. Nanoparticles may also optionally comprise myeloid, macrophage or microglia-specific surface ligands to enhance their transfer into myeloid, macrophage or microglia cells. Nanoparticles may optionally comprise surface ligands that enhance their transfer to target cells such as neurons, astrocytes, oligodendrocytes, and endothelial cells of the brain. Suitably, nanoparticles may be at least 20 nm, at least 25 nm, at least 35 nm, at least 50 nm or at least 75 nm in average diameter. Suitably, nanoparticles may be less than 600 nm, less than 500 nm, less than 300 nm, less than 250 nm, less than 200 nm, less than 150 nm or less than 100 nm in average diameter. Suitably, nanoparticles are of a size that does not induce an inflammatory response in the target cell.

Liposomes may also be suitably used to encapsulate a therapeutic agent and used instead of, or in addition to, nanoparticles in the methods and compositions of the present invention.

As used herein, a “therapeutic agent” is an agent, or combination of agents, that treats a cell, tissue or organism having a condition requiring therapy, when contacted with the cell, tissue or organism. The therapeutic agent may be suitably encapsulated, adsorbed or attached to the nanoparticle. Non-limiting examples of suitable therapeutic agents include small molecules, drugs, polypeptides, antagomirs, cytotoxic agents, chemotherapeutic agents, anti-angiogenic agents, radioactive agents, imaging agents, cytokines, growth factors, apoptotic pathway effectors, neurotransmitter precursors, neurotransmitter agonists or antagonists, antibodies, radionuclides, reactive oxygen species scavengers, anti-inflammatory agents, analgesics or polynucleotide sequences, such as RNAi-inducing agents, siRNA, antisense RNA, miRNA (micro RNA) or DNA or combinations thereof. Suitably, the polynucleotide may be expressed in the cell, tissue, or organism requiring therapy, or in MMMsc cells used to deliver the therapeutic agent.

As used herein, an “RNAi-inducing agent,” encompasses RNA molecules or vectors whose presence within a cell results in RNA interference and leads to reduced expression of a transcript to which the RNAi-inducing entity is targeted. The term specifically includes short interfering RNA (“siRNA”), short hairpin RNA (“shRNA”), and RNAi-inducing vectors. Selection of appropriate target sequences for RNAi may take into account factors such as synthetic considerations, avoidance of targeting unwanted transcripts, and other considerations, as described by Manoharan, Current Opinion in Chemical Biology 2004, 8:570-579 (2004), which is incorporated herein by reference in its entirety.

A “cytotoxic agent,” as used herein, refers to any agent that induces apoptosis, anoikis or necrosis in cells to which the cytotoxic agent is delivered. In the context of brain cancers, cytotoxic agents such as temozolomide may be particularly suitably delivered. Other suitable cytotoxic agents include particular chemotherapeutic moieties, as described herein. A “chemotherapeutic agent,” as used herein, refers to any agent that interferes with cell division, disrupts normal functionality of microtubules, inhibits utilization of a metabolite, substitutes nucleotide analogs into cellular DNA, or inhibits enzymes necessary for DNA replication.

If the therapeutic agent comprises a polynucleotide comprising a coding sequence encoding a polypeptide or RNA, the coding sequence may be suitably operably linked to a promoter. Suitable promoters include but are not limited to, constitutive promoters, such as CMV, inducible promoters and cell-specific promoters. Suitable cell-specific promoters include, but are not limited to, astrocyte-specific promoters (e.g. GFAP) glioma promoters (e.g. human glial fibrillary acidic protein promoter gfa2; midkine-promoter), oligodendrocyte promoters (e.g. myelin basic protein (MBP) promoter), and endothelial cell promoters (e.g. synthetic promoters described in Dai et al., Journal of Virology 78: 6209-6221, (2004) Identification of Synthetic Endothelial Cell-Specific Promoters by Use of a High-Throughput Screen, herein incorporated by reference in its entirety).

In one embodiment, the polynucleotide may be associated with a polynucleotide encoding a reporter polypeptide, such as a fluorescent protein such as GFP. Suitably, if the polynucleotide encodes a therapeutic polypeptide, a fusion protein comprising the reporter polypeptide and the therapeutic polypeptide may be expressed in the MMMsc cells or cells of the target tissue, organ, or in vitro cell culture. Additionally, if the therapeutic polynucleotide encodes a polypeptide, an RNAi-inducing agent, siRNA, antisense RNA, or miRNA, expression of the reporter polypeptide may indicate the presence or expression of the therapeutic polynucleotide.

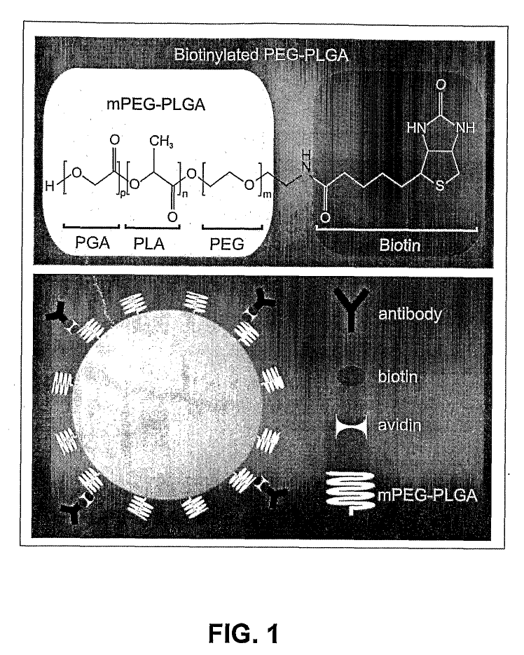

Nanoparticles may be synthesized using any method described in the art, or disclosed herein. In one embodiment, nanoparticles are modified by polyethylene glycol (PEG) conjugation, a process known in the art as “PEGylation.” A suitable structure of a PEGylated nanoparticle is shown in FIG. 1. Nanoparticles may be suitably made using polygycolic acid (PGA) and poly-lactic acid (PLA) to form a copolymer (FIG. 1, top) used to construct the nanoparticle matrix (FIG. 1, bottom). The immunogenicity of the nanoparticles may be suitably reduced by incorporation of methoxypolyethylene glycol (mPEG). Suitably, the nanoparticles comprise poly (lactic-co-glycolic acid) (PLGA), mPEG or a combination thereof. Antibodies may be suitably attached via avidin-biotin bridges to enhance uptake of the nanoparticles. PEGylation of nanoparticles may reduce their uptake into macrophages, myeloid cells and microglia. However, in one embodiment of the present invention, endocytotic uptake of nanoparticles into MMMsc occurs in vitro, away from the in vivo mononuclear phagocyte system. Nanoparticle uptake and transfection into specific cells may also be optionally enhanced by complexing PEG-based nanoparticles to ligands specific to isoform subtypes of receptors found primarily on MMMsc. PEG surface density also may be optionally reduced to uncover the hydrophobic and charged polymeric core, which may substantially increase endocytosis by MMMsc. In addition, the PEG carrier may be hydrolyzed to expose the hydrophobic core of the nanoparticle, and facilitate its subsequent uptake into MMMsc.

Nanoparticles may suitably be modified to affect the release rate of the therapeutic agent, biodegradation rate of the nanoparticles, and nanoparticle size. For example, a water-in-oil-in-water (w/o/w) double emulsion may be used where the desired polynucleotide is contained in an aqueous buffer solution inside an oily bubble containing a volatile solvent and the dissolved polymer. This mixture may be suspended in an aqueous solution. Evaporation of the volatile solvent may produce a hardened polymer shell containing the aqueous solution of the desired polynucleotide and may contain the lactide-glycolide polymer covalently bound to PEG. This copolymer may release the therapeutic agent over days to weeks. Suitably, a second method utilizes long chains of PEG alone and may increase the polynucleotide payload and increase the release rate of the polynucleotide. In each case, functionality may be introduced via a biotin end group on the PEG. Suitably, nanoparticles made from mPEG-PLGA are not immunogenic and are non-toxic. PLGA is an FDA approved material used in, among other things, resorbable suture material. A suitable structure of an m-PEG-PLGA nanoparticle is shown in FIG. 1. In vivo, PLGA polymers may be hydrolyzed (to lactic acid and glycolic acid) and then metabolized via the Krebs cycle to carbon dioxide.

Nanoparticles carrying polynucleotide coding sequences may be taken up by MMMsc cells in primary cultures or by MMMsc cells isolated from blood or tissue samples. Primary cultures of monocytes or bone marrow stem cells may be prepared from blood or bone marrow using techniques known in the art, such as involving the “adherence” method “adherence” method. Primary cultures of macrophages may be obtained from blood using techniques known in the art, such as the adherence method. Primary cultures of microglial cells may be obtained from hippocampal tissue. Alternatively, microglia can be differentiated from myeloid cells or macrophages by exposure to brain tissue, astrocytes or their media. In addition, other immune cells (e.g., T cells, B cells, dendritic cells) or stem cells can be differentiated to increase their tendency to track to brain and inflammation there.

In one embodiment, macrophages may be loaded ex vivo in long-term culture with nanoparticles carrying one or more therapeutic agents. The long-term culture permits a high transfection efficiency. As used herein, “long term culture” means culturing for at least 5 days at least 10 days, at least 15 days, at least 20 days, at least 25 days or at least 30 days.

In one embodiment of the invention, uptake of nanoparticles by MMMsc may be carried out by incubating MMMsc with the nanoparticles under conditions that permit endocytosis of the nanoparticles. Endocytotic uptake may be suitably carried out by adding nanoparticles to normal growth media over MMMsc cells for a sufficient period of time (e.g., one or more hours, or continuously) to permit movement from endosomes to the cytosol. Suitably nanoparticles are provided at concentrations that are non-toxic or not high enough to trigger cell death. Optionally, bone marrow stem cells, monocytes or macrophages may be differentiated via culturing to monocytes, macrophages or microglia before or after being contacted with nanoparticles.

Suitably the MMMsc are incubated under conditions that maintain nanoparticles in the cell, or that minimize or prevent exocytosis or phagocytotic degradation of the nanoparticles. The maintenance of nanoparticles in the MMMsc may be suitably accomplished by holding the MMMsc in a medium comprising nanoparticles. Nanoparticle-loaded MMMsc may also be incubated in media that is free or substantially free of serum or protein to maintain nanoparticles in the MMMsc. As used herein, “substantially free of serum or protein” means that the media contains only trace levels of extracellular serum or protein. Suitably, the media is at least 97%, at least 98%, at least 99%, at least 99.5% or at least 99.9% free of serum or protein. MMMsc may also be suitably held in a medium sufficient to prevent or inhibit exocytosis of the nanoparticles, or to amplify or enhance the inhibitory effect of protein removal. Suitable media, include, but are not limited to saline media, hypotonic media, hypertonic media or acidic media. Cells maintained in a serum-free medium (e.g., DMEM comprising 0.1% gentamycin) may retain at least 50%, at least 60%, at least 75%, at least 80%, at least 85%, at least 90% or at least 95% of nanoparticles for a period of at least 1 hour, at least 2 hours, at least 3 hours, at least 5 hours, at least 6 hours, at least 8 hours, at least 12 hours, at least 16 hours, at least 20 hours, at least 24 hours, at least 36 hours, at lest 48 hours or at least 72 hours.

In one embodiment, nanoparticles may be taken up by MMMsc cells via endocytosis including, but not limited to, phagocytosis, pinocytosis, clathrin-dependent receptor mediated endocytosis, and clathrin-independent endocytosis. Polynucleotides attached to or encapsulated by nanoparticles may be released to drive protein expression, for example, for days, weeks, or months in an MMMsc cell, a neuronal cell culture or in a target brain cell in vivo.

MMMsc cells comprising nanoparticles carrying polynucleotides may suitably express the polynucleotide. The polynucleotide may be expressed episomally, or may be incorporated into the genome of the macrophage. The expression product may be suitably delivered to brain, neural tissue or neural cultures. In one embodiment, the nanoparticle carrying a polynucleotide is delivered from the MMMsc and taken up by another brain cell, either via endocytosis, or any other suitable mechanism. The polynucleotide may be expressed episomally in the other brain cell, or may be incorporated into the genome of the other brain cell.

MMMsc cells comprising nanoparticles comprising therapeutic agents may be tested using hippocampal organotypic cultures (HOTCs), or in whole animals. HOTCs are a widely accepted in vitro model of brain tissue, are similar to their in vivo counterparts and are stable. Suitably, the MMMsc comprising the nanoparticles may be harvested for use as biological vehicles for delivery to HOTCs, to whole animals or mammals via, for example, intranasal or intravascular injection, or by direct injection into brain lesions. The incorporation of polynucleotide sequences into nanoparticles may also be used to alter MMMsc surface protein expression to regulate their penetration past the blood-brain barrier.

Nanoparticles may be suitably evaluated for loading efficiency of a polynucleotide therapeutic agent, using polynucleotide sequences encoding, for example, green fluorescent protein (GFP) to identify transfected cells expressing GFP. Digital fluorescent imaging of GFP cells may indicate transfection efficiency. Suitably, gene encoded fluorescent indictor (GEFI) for free cytosolic calcium (Ca²⁺ᵢ) may be used to monitor the functional state of transfected cells. Tumor necrosis factor alpha (TNF-α), may also be suitably expressed conditionally to evoke endogenous neuroprotection. For example, temporal lobe epilepsy may be used as an exemplary model of brain disease and induced using kainic acid in both HOTCs and whole animals. Other imaging techniques may be used to confirm vitality of microglial cells and adjacent neural tissue following transfection and induction of temporal lobe epilepsy. Potential inflammatory changes of transfected cells and surrounding tissue may be measured using real-time RT-PCR of specific-cell-enhanced samples and tissue obtained via laser dissection microscopy.

Nanoparticles, contained within MMMsc cells or released from MMMsc cells into the body, may be tracked in the body using any technique known in the art, including, but not limited to, incorporating small amounts of nanocrystalline magnetic into the nanoparticles and imaging using MRI, dissolving organic chelators, such as diethylenetriaminepenta acetate, within the polymer in the presence of radioactive transition metals (e.g. ion-59, yttrium-90, indium-111) or lanthanides (e.g., europium-152, europium-154) and tracking by gamma imaging.

MMMsc cells comprising nanoparticles comprising a therapeutic agent may be targeted to the brain of a mammal and used to treat a variety of disease conditions of the brain. In healthy brain, signals from the immune system enhance and preserve learning and memory. However, in neurological disease, similar immune signaling may turn lethal and so worsen the severity of associated brain injury.

Suitably, for the treatment of a disease of a mammal, the MMMsc cells that will comprise a therapeutic agent carried by a nanoparticle may be extracted from blood or bone marrow samples of the mammal suffering from the disease or condition, or a blood or bone marrow sample from a matched donor, such that the MMMsc cells delivered to the mammal in need of treatment are autologous or matched.

Methods and compositions of the invention may also be used to deliver therapeutic agents in combined therapies. As used herein, “combined therapies” is intended to include multiple therapeutic agents administered at a single treatment time, multiple therapeutic agents administered singly or in combination over time, or a combination thereof.

In one embodiment of the invention, the delivery of MMMsc carrying nanoparticles to the brain may be enhanced by increased neural activity generated, for example, by stimulant treatment. Treatments may suitably guide the MMMsc carrying nanoparticles to target regions of the brain, neural tissue or neural culture. For example, increased neural activity (without tissue injury) may trigger a release of inflammatory mediators that may be used to enhance the movement of macrophages containing nanoparticles carrying a therapeutic agent into diseased brain, as well as surrounding normal brain. Stimulant treatment includes, but it not limited to, spreading depression, increased neural activity evoked by intravascular stimulants, brain magnetic stimulation, peripheral nerve stimulation or increased sensory input. Spreading depression is a short-lasting, non-injurious depolarization wave that moves across the brain, neural tissue or neural culture. Spreading depression shows many of the biophysical, biochemical and cellular changes of ischemia. While benign, spreading depression is a robust perturbation of susceptible brain areas that triggers long-lasting inflammatory changes, including astrogliosis, microgliosis, increased expression of cyclooxygenase-2 and innate cytokines. These inflammatory changes can be expected to occur with changes in chemokines. Spreading depression may be suitably induced by mechanical stimulation, electrical stimulation, chemical stimulation (e.g., using KCl) of the brain, neural tissue or neural culture.

Methods and compositions of the invention may be used to treat a wide range of neural diseases or conditions, such as acute, sub-acute and chronic neurodegenerative diseases, including, but not limited to, Parkinson's disease, stroke, neonatal stroke, traumatic brain injury, Huntington's disease, Alzheimer's disease and carbon monoxide exposure. Each of these conditions is characterized by excitotoxic injury and suitable treatments may be evaluated using standard animal model systems as described in Examples 18, 19 and 20. Suitable treatments include, but are not limited to, the administration or delivery of MMMsc cells comprising nanoparticles comprising a suitable therapeutic agent. Suitably, the cells comprising nanoparticles comprising a therapeutic agent may be administered to the brain of a mammal suffering from the neural disease or condition via intra-arterial injection, intra-thecal injection, intra peritoneum injection, intra-nasal injection or intra-venous injection.

Suitable therapeutic agents, include, but are not limited to, antioxidants (e.g., Cu/Zn SOD, glutathione), inhibitors of apoptotic genes (e.g., inhibitors of pro-apoptotic p53, Bad), anti-apoptotic polypeptides (e.g., Bcxl-2 family), and modulators of transcription factors (e.g., NFkB) and kinases (e.g., phosphorylinositol 3-kinase (PI3K), phospholipase C (PLC), protein kinase (PKC) pathways, MAP kinase pathways, ERK 1/2 associated with apoptosis and anti-apoptosis, and enhanced growth factors (e.g., insulin growth factor 1, nerve growth factor, fibroblast growth factor-2, brain derived neurotrophic factor). Other suitable therapeutic agents include those which alter the magnitude, duration, or extent of the inflammatory response (e.g., by control of TNF-α, IL-1B, and other cytokines). Suitable therapeutic agents also include polynucleotides encoding these therapeutic agents or polynucleotides modulating the expression of polypeptides or polynucleotides (such as effectors) involved in the disease processes.

Other neuronal diseases and brain disorders suitable for treatment using methods and compositions of the invention include, but are not limited to, primary or metastatic brain tumors, chronic pain, auto-immune disorders (e.g. multiple sclerosis), amyotrophic lateral sclerosis and epilepsy seizures. For example, brain tumor cell-specific delivery of nanospheres carried in macrophages and containing interference RNA (RNAi) for epidermal growth factor receptor (EGFR) may be used to reduce disease severity from primary glioma and tumor burden. Nanospheres carried in macrophages may contain a plasmid to enhance cellular release of interleukin-10 and be used in methods of the invention to lessen chronic pain, with the process optionally augmented by peripheral nerve, cranial nerve or central nervous system stimulation. In another embodiment, nanoparticles carried in macrophages and containing a plasmid to enhance cellular release of galanin may also be used to mitigate the impact of temporal lobe epilepsy. In a further embodiment, nanoparticles carried in macrophages and containing a plasmid to enhance cellular release of fibroblast growth factor-2 (FGF-2) may ameliorate the impact of multiple sclerosis, for example, experimental multiple sclerosis.

Other diseases and conditions may be amenable to treatment with methods and compositions of the present invention including, but not limited to cancer, type 1 diabetes, amelioration of transplant rejection, heart disease, renal diseases, liver disease, rheumatoid arthritis, systemic lupus erythematosus, and focal infectious diseases. Suitably, these diseases may be treated by delivering macrophages comprising nanoparticles carrying one or more therapeutic agents, for example, a polynucleotide, to the target disease organ or tumor. The macrophage may then suitably release nanoparticles comprising the therapeutic agent for uptake by the cells of the target disease organ or tumor.

### EXAMPLES

### Example 1

### Large-Scale Manufacture of Polynucleotides Encoding a Green Fluorescent Protein (GFP)

Plasmid DNA (pEGFP-N1; from Clontech, GenBank Accession #U55762) was isolated from bacteria using the EndoFree Qiagen plasmid maxi purification kit following the manufacturers protocol (from Qiagen). pEGFP-N1 includes a polynucleotide encoding EGFP under the control of CMV, a constitutive promoter. To produce larger quantities bacteria comprising the pEGFP-N1 plasmid were grown in 100 mL LB batches.

### Example 2

### Nanoparticle Fabrication

PLGA and mPEG-PLGA nanoparticles of uniform size (i.e., 100 nm, 150 nm and 250 nm) were synthesized. PLGA nanospheres (of approximately 250 nm diameter) were produced as follows. 4 mg acetylated bovine serum albumin (BSA) (Sigma) was dissolved in 66.7 μL of water. 400 μg DNA (pEGFP-N1) was dissolved in 40 μL TE 7.4 buffer. 12 mg of PLGA (MW: 30,000; 50:50 (from Boehringer Ingelheim)) polymer was dissolved in 400 μL chloroform. If nanoparticles capable of carrying specific surface monoclonal antibodies (mAbs) were desired, 1-2% of the starting mPEG-PLGA (or PLGA) polymer was replaced by biotin-PEG-PLGA (or biotin-PLGA). The DNA and BSA were added together to the dissolved PLGA with a glass Pasteur pipette while the PLGA was sonicated. The emulsion was sonicated for 30 seconds at 45-50 watts in an ice bath. The primary water/oil (w/o) emulsion was added to 3.33 mL of 2.5% polyvinyl alcohol (PVA); MW: 50,000) with a glass Pasteur pipette while the PVA was sonicated. The emulsion was sonicated for 1 min at 45-50 watts in an ice bath. The w/o/w emulsion was stirred overnight at 500 rpm. The nanoparticles were washed by centrifugation. Nanoparticles were spun at 25,000 rpm for 20 min at 4° C. The nanoparticle pellet was then washed twice with 3 mL 1% PEG 400 and resuspended in 1 mL 1% PEG 400, or lyophilized and stored at −80° C. Some aggregation of nanoparticles was noted.

In a second fabrication method, 4 mg acetylated BSA (Sigma) was dissolved in 66.7 μL water. 400 μg DNA (pEGFP-N1) was dissolved in 40 μL TE 7.4 buffer. 12 mg of PLGA polymer (MW: 30,000; 50:50 (from Boehringer Ingelheim)) was dissolved in 400 μL acetone. If nanoparticles capable of carrying specific surface monoclonal antibodies (mAbs) were desired, 1-2% of the starting mPEG-PLGA (or PLGA) polymer was replaced by biotin-PEG-PLGA (or biotin-PLGA). The DNA and BSA were added together to the PLGA with a 1 mL syringe with a 22 G 1½ needle within the liquid while stirring at 500 rpm. The primary w/o emulsion was added to 3.33 mL 2.5% PVA with a syringe with a needle within the liquid while stirring at 500 rpm. The w/o/w emulsion was stirred overnight at 500 rpm. A stirred cell filtration unit (Amicon 8200 with YM100 membrane) was used to rinse out the PVA. Nanoparticles were washed twice with 1% PEG 400, using 10 times the volume of the nanoparticles. The final wash was filtered to give a final volume of 1 mL 1% PEG 400. Nanoparticles were stored in 1% PEG or were lyophilized and stored at −80° C.

For GFP-DNA plasmid loading of nanospheres, 1 mg of DNA encoding GFP and 2 mg nuclease-free acetylated bovine serum albumin in 200 μL of TE buffer was emulsified into the polymer solution to give a DNA loading concentration of >40 μg/mL. To produce a strong burst release of DNA within the first several hours of transfection, the second aqueous phase containing the PVA was saturated in DNA solution.

For coumarin-loaded nanoparticles, nanoparticles were fabricated via the two methods described above except that rather than using DNA, the fluorescent marker 6-coumarin (Sigma) was dissolved in acetone (1:6,000 w/w coumarin/PLGA).

For nanoparticles biotinylated to carry antibodies (mAb), the mAb were also biotinylated. An EZ-Link Sulfo-NHS-Biotin kit (from Pierce) was used according to the manufacturer's directions and LaRochelle W J and Froehner S C (Determination of the tissue distributions and relative concentrations of the postsynaptic 43-kDa protein and the acetylcholine receptor in Torpedo. J Biol Chem. 1986 Apr. 25; 261(12):5270-4). mAb 2 mg/ml in 100 mM sodium bicarbonate (pH 8.0) were combined with sulfosuccinimido-biotin according to manufacturer's recommendations and were incubated for 4 hours (25° C.). Non-reacted biotin was removed by dialysis in phosphate buffered saline (PBS). Biotinylated mAb and nanospheres were combined via an avidin bridge. A mAb directed against the transferrin receptor (OX26; Abcam) was used to enhance entry of nanospheres to myeloid cells. Similarly, a monoclonal antibody directed against excitatory amino acid transporter (EAAT) subtypes was used to enhance relative delivery of nanospheres to either neurons (EAAT-3; Sigma) or astrocytes (EAAT-2; Sigma). Similar cell-surface-specific chemistry was used to target nanospheres selectively to oligodendrocytes.

### Example 3

### Characterization of Nanoparticles

Nanospheres were examined using scanning electron microscopy (SEM) to illustrate the size of nanospheres. Samples were prepared by dropping an aliquot of the nanosphere suspensions of Example 1 onto a polished aluminum stub. Excess solution was wicked off with filter paper leaving a thin coating of nanospheres. A thin layer of a gold coating covering the particles was applied by sputtering gold at 10 mA and 6 V (Model Huml; Technics) for 0.5 to 6 minutes. The surface morphology of the nanospheres was examined by High Resolution SEM (Hitachi S-4700-II) at 5-15 kV. The particles ranged in size from about 200 nm to about 250 nm, appeared round and showed little evidence of aggregation.

Particle sizing, particle distribution, and zeta potential measurements were performed using a Brookhaven ZetaPlus Analyzer. Nanoparticle size standards from Duke Scientific were prepared according to the certification records and used to check the performance of the system. A zeta potential standard, B1-ZR3, was prepared according to the manufacturer's instructions. Aliquots of nanoparticle suspensions were diluted with deionized water for sizing measurements to eliminate multiple scattering. Solutions for zeta potential measurements were adjusted for pH using 0.1 M nitric acid or sodium hydroxide. Multiple measurements were made to determine the average size and zeta potential. The size and zeta potential of the nanoparticles of Example 2 correlated with the certified values.

Nanoparticle yield was measured for 250 nm nanospheres. A nanoparticle suspension was removed from its vial and the residual polymer weighed to determine losses during the synthesis. The nanoparticle suspension was washed three times with filtered deionized water and recovered by centrifugation with an ultracentrifuge at 40,000 rpm and 4° C. for 15 min. The nanoparticles recovered by centrifugation were lyophilized for 24 hours, and then weighed. The supernatant was analyzed by light scattering and the count rate was used to determine losses from washing and centrifugation. The total polymer recovered and lost from five batches was compared to the theoretical amount of nanospheres for each batch and the average yield expressed as nanosphere production efficiency of about 50%.

DNA plasmid encapsulation efficiency was determined from the quantity of plasmid DNA in the nanospheres compared to the starting concentration (1 mg/200 μL). An aliquot of nanospheres was placed in 10 mL of 0.05 N NaOH/0.5% sodium dodecylsulfate for 16 hour. This method was supplemented by the residual method where the concentration of DNA in the starting solution (1 mg/200 μL) was compared to the final solution concentration after nanosphere formation and included the concentration of DNA in the wash solutions. The DNA content was quantified using the PicoGreen™ (Invitrogen) assay and via real-time PCR for more accurate quantitative measurements. On average, about 29% DNA encapsulation efficiency was seen.

### Example 4

### Release of Plasmid DNA Incorporating a Polynucleotide Encoding GFP from PLGA Nanospheres

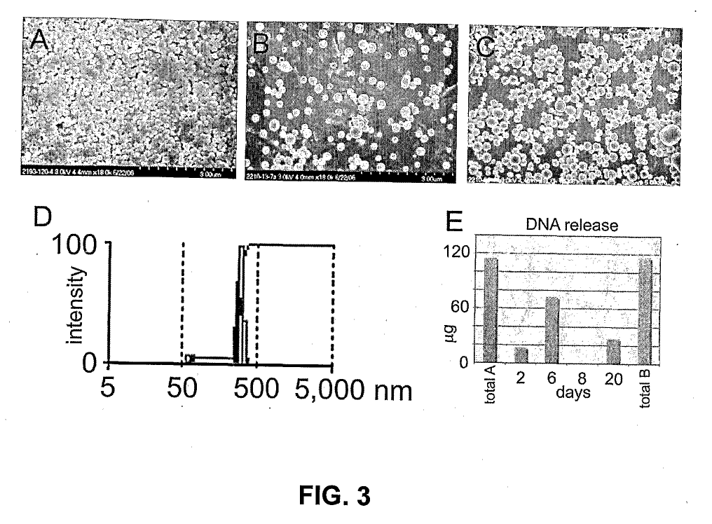

Empty PLGA nanospheres, coumarin-labeled nanospheres and GFP-plasmid DNA containing nanoparticles were produced according to the procedures described in Example 2. Scanning electron micrographs were taken of empty nanospheres (FIG. 3 A), coumarin-loaded nanospheres (FIG. 3 B) and GFP-plasmid containing nanospheres (FIG. 3 C). The particles in each of the samples were of uniform size (approximately 250 nm) and evenly dispersed with little evidence of aggregation. Light scattering measurements of nanoparticles containing GFP showed a uniform diameter of 245.5±1.4 nm (n=6 measurements); polydispersion of 0.146; average count rate of 0.146 Mcps; and sample quality of 9.2 (FIG. 3, D). FIG. 3, E shows the amounts of DNA released from the nanoparticles at day 2, day 6, day 8 and day 20 as an increment of change from the previous reading. The rate of release of DNA from the GFP-loaded nanoparticles into free solution at 25° C. was highest for the first 6 days, and continued for at least 20 days (FIG. 3, E). Total DNA incorporated into the aliquot of nanospheres (total A) was 114 μg and the sum total released over a period of 20 days (total B) was 116 μg, within the error of individual measurements (FIG. 3, E). These results show that DNA from PLGA nanoparticles continued for at least 20 days.

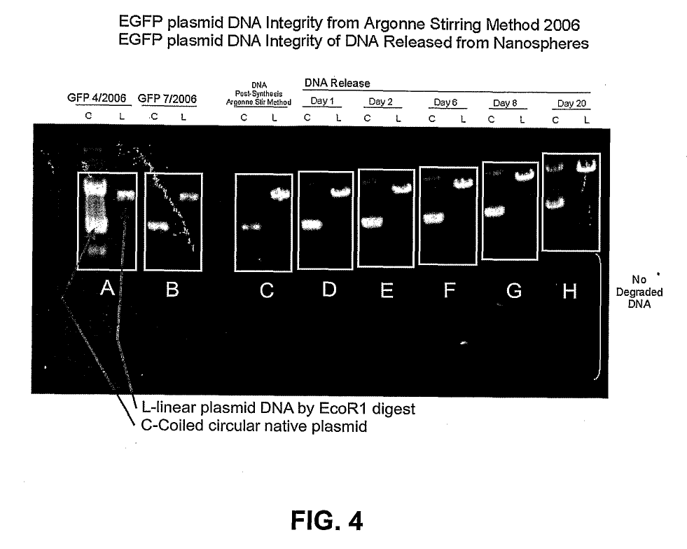

The integrity of the released GFP-plasmid DNA from the nanoparticles was also tested. Nanoparticles were produced using the non-aggregating method of Example 2, which did not include sonication steps. Two control samples were used GFP-plasmid DNA that was used to prepare the nanoparticles and GFP-plasmid DNA left in solution following its incorporation into nanoparticles. DNA released into free solution on days 1, 2, 6, 8 and 20 days of incubation of the nanoparticles at 25° C. and the control samples was untreated or linearized with EcoR1, and analyzed by agarose gel electrophoresis (FIG. 4). Both super-coiled native GFP-plasmid DNA and GFP-plasmid DNA linearized with EcoR1 were measured for all samples. The integrity of GFP-plasmid DNA released at days 1, 2, 6, 8 and 20 was analogous to that of the control sample used to prepare the nanospheres. No degraded DNA was evident in any of the samples.

### Example 5

### Preparation of Bone Marrow and Blood Macrophages and Primary Microglia

Wistar rats were euthanized by blood withdrawal via cardiac puncture under pentobarbital anesthesia to harvest blood via cardiac puncture and then decapitated to harvest bone marrow. Bone marrow was collected via aspiration from tibia and femur bones. Mononuclear cells were isolated by density gradient centrifugation and then plated in culture media containing colony-stimulating factor for 24 hours. Cells adhered after 24 hours were marrow stromal cells and mature macrophages and were removed. Non-adherent cells were transferred to new flasks, or 25 mm diameter glass coverslips for cell cultures placed individually in wells of 6-well culture plates, with EMEM-10 media containing growth factor and grown for 4 days. Another supplement of EMEM-10 was added and growth continued for another 3 days before use. For long-term use, bone marrow macrophages were fed daily with a half-media change using astrocyte-conditioned media. These procedures produced macrophages that were able to take up GFP-polynucleotide containing nanoparticles and express a GFP polynucleotide payload over weeks.

Microglia were prepared from 0-2 day old rat pup hippocampus as previously described following anesthesia with 100% CO₂ and decapitation according to Caggiano A O and Kraig R P, Prostaglandin E2 and 4-aminopyridine prevent the lipopolysaccharide-induced outwardly rectifying potassium current and interleukin-1beta production in cultured rat microglia, J Neurochem. 1998; 70(6):2357-68, incorporated herein by reference in its entirety. Microglia were ready for use approximately 14 days after culture. For long-term use, microglia were fed daily with a half-media change using astrocyte-conditioned media, i.e. media (DMEM, 10% fetal bovine serum, 0.1% gentamycin) drawn from astrocyte cultures and comprising molecules secreted by astrocytes. These procedures produced microglia that were able to take up GPF-polynucleotide containing nanoparticles and express a GFP polynucleotide payload over weeks.

### Example 6

### Loading of Nanoparticles into Microglia and Macrophages

Microglia were loaded with polynucleotide- or coumarin-loaded nanoparticles by incubation of a microglial cell culture with the nanoparticles for 1 hr or 3 hr in normal (DMEM, 10% fetal bovine serum, 0.1% gentamycin) media or continuously (i.e., greater than a week) in normal media half-changed daily with astrocyte-conditioned media.

Macrophages were similarly loaded.

### Example 7

### Uptake of Coumarin-loaded Nanoparticles by Microglia and Macrophages Via Endocytosis

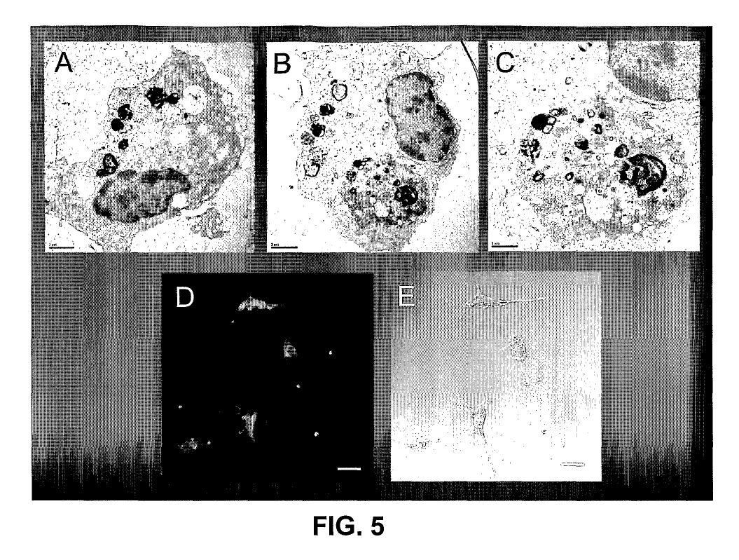

Transmission electron micrographic (TEM) images of microglia in primary culture and of microglia following exposure to coumarin-containing nanospheres (of 188 nm diameter) at 100 μg/mL for one hour are shown in FIG. 5, A, B and C (calibration bar is 1 μm in A and B and 2 μm in C). A normal microglial cell in primary culture showed a typical nucleus (red arrow) and a diverse array of vacuoles (light areas) and so-called “dense bodies.” (FIG. 5, A). Microglia retained this typical appearance after exposure to 100 μg/mL coumarin containing nanoparticles of 188 nm diameter (FIG. 5, B). A high power image of the cell shown in B exhibited uniformly sized nanospheres (yellow arrows) within endosomes (FIG. 5, C). Confocal images further confirmed microglial uptake of fluorescent coumarin nanoparticles, which give off a green light when excited at 488 nm. Microglia were pseudo-colored with a “fire” LUT from Image J software to indicate fluorescence intensity, and four representative cells, fluorescing red, are shown in FIG. 5, D (calibration bar 20 μm). The morphology of the four fluorescent microglial cells was imaged using transmitted light, and is shown in FIG. 5, E (calibration bar 20 μm).

### Example 8

### Expression of GFP in Microglia and Macrophages Transfected with Nanoparticles Encapsulating GFP-Encoding Plasmid DNA

GFP expression was examined using routine fluorescence microscopy (and semi-quantitative fluorescence intensity measurements). For improved sensitivity, GFP expression was analyzed with bright field microscopy after immunostaining (using horse radish peroxidase coloration) for GFP expression. For presentation of morphological characteristics, selected cells were visualized using confocal microscopy.

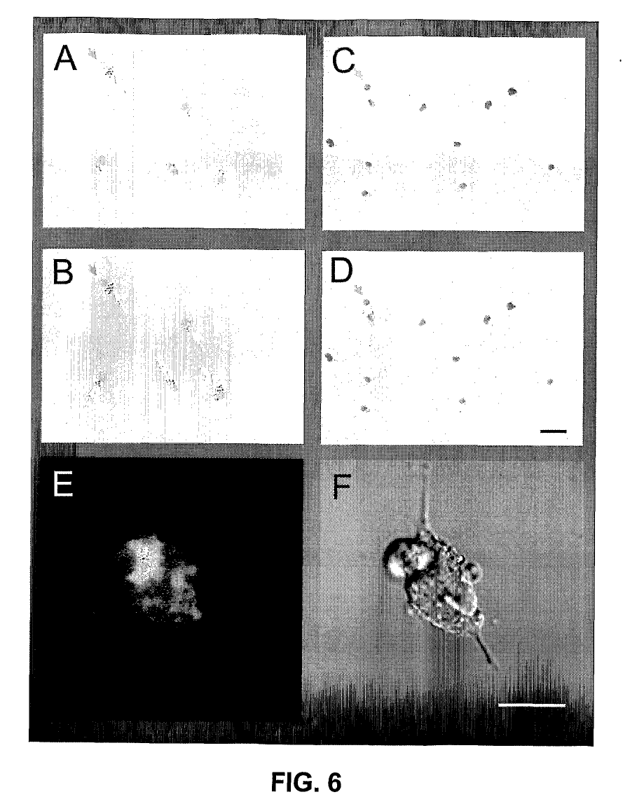

Nanoparticles were made according to Example 2, using the second method with less aggregation of the particles, from 444 μg GFP-DNA plasmid that was encapsulated in a batch of 3 mg PLGA. Images were taken using bright field and confocal microscopy. Evidence of GFP protein expression in microglia was faintly evident using fluorescence microscopy even after 6 hours post transfection, and was more evident by 1 and 3 days post transfection. However, background fluorescence of microglia was found to hamper distinction between GFP protein positive and negative cells via fluorescence microscopy. Accordingly, GFP protein expression was evaluated using bright field immunostaining for GFP protein (using an antibody to GFP (Beckton-Dickinson)) and a horse radish peroxidase secondary antibody that stains positive as the color “brown.” Toluidine blue counterstaining was used to identify nuclei of microglia cells (see red arrows of FIG. 6, A-D). GFP positive stain was evident as brown punctuate staining in microglia and macrophages transfected with nanoparticles containing the polynucleotide encoding GFP using bright field microscopy (FIG. 6, A). The staining was enhanced for quantification using Nomarski optics (FIG. 6, B). Control cells containing PLGA nanospheres without GFP-DNA plasmid did not show any evidence of punctuate brown staining using normal bright field optics (FIG. 6, C) or Nomarski optics enhancement (FIG. 6, D). Indeed, quantification of 125 transfected cells showed 106 positive for GFP protein and 19 negative (quantification efficiency of 85%). In contrast, control cells exposed to nanospheres without DNA never showed positive brown punctuate staining (transfection efficiency of 0%; n=112).

Fluorescence from positive GFP protein expression was also detectable using confocal fluorescence microscopy. Microglia and macrophages were exposed to nanospheres at 444 μg/mL for 3 hours. Confocal fluorescence microscopy images of fluorescing transfected cells were obtained using Image J software and pseudo coloring the images with “fire” LUT. Red fluorescence was clearly evident within the cells.

### Example 9

### Long-Term Exposure to GFP-Plasmid Nanoparticles Enhanced GFP Expression in Microglia

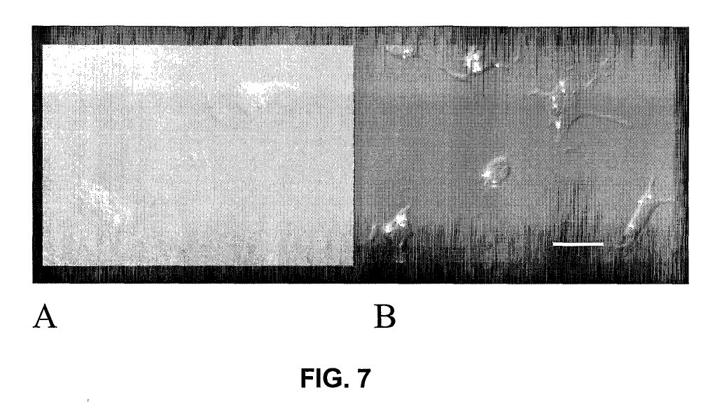

Microglia were cultured according to the method described in Example 5, with a daily half-change of astrocyte-conditioned media, and were kept alive for weeks. Cells exposed to GFP-plasmid DNA carrying nanospheres (initial concentration of 20 μg/mL) continued to proliferate during the culture period and became virtually confluent. The long culture period enabled microglia to express a high level of GFP protein. Continued exposure to GFP-plasmid carrying nanospheres triggered a progressively greater level of green fluorescence in cells, evident with routine fluorescence microscopy and cellular proliferation, as shown in FIG. 7. Fluorescence microscopic images of GFP were pseudo-colored with a “fire” LUT from Image J software to indicate fluorescence intensity. Nomarski images of the same cells were taken and showed morphological detail and cellular sites of GFP protein. After 3 days of culture with the GFP-plasmid DNA, only mild fluorescence was evident in all cells (FIG. 7 A). However by 8 days in culture, fluorescence of GFP in all microglial cells was dramatically greater (FIG. 7 B). Furthermore, many more cells per observation field were noted (FIG. 7 B) indicating continued proliferation of the microglia.

### Example 10

### Green Fluorescent Protein Polynucleotide Transfection of Hippocampal Organotypic Culture Cells

Hippocampal Organotypic Culture (HOTC) neural cells were transfected with a polynucleotide encoding green fluorescent protein (GFP) by biolistic introduction of the pEGFP-N1 vector coated onto the surface of gold microparticles. The transfection triggered GFP protein expression in neural cells. The expression of GFP was monitored. Images taken of the transfected cells showed GFP-positive hippocampal pyramidal neurons in a HOTC. Superimposition of microglial appearance from a separate HOTC stained red with fluorescently labeled DiLDL revealed that the microglia are evenly distributed within gray matter of brain. The even distribution of microglia in resting brain, coupled to their natural ability to move makes these cells well-suited to act as reservoirs for delivery of therapeutic materials to brain.

### Example 11

### Culture and Induction of Spreading Depression in Hippocampal Organotypic Culture Cells

HOTCs were prepared and maintained by initial incubation in serum-based media followed by transfer to a serum-free media (SFM) at 4 days in vitro (DIV). The transfer from serum-containing medium to SFM prevented neuronal loss from occurring after two weeks of culture (stability was measured by neuron-specific nuclear protein (NeuN)-based western blots), and prevented glial activation in the HOTCs. SFM contained Neurobasal medium ((96%) from Invitrogen) supplemented to 42 mM glucose, 1 mM glutamine (from Sigma), 2% B27 supplement, 10 μg/mL gentamicin and 250 μg/mL Fungizone (from Invitrogen). Gentamycin was used rather than penicillin in the SFM, because penicillin adversely affected HOTCs such that by 21 DIV, most pyramidal cells were dead and no CA3 field potentials were seen from dentate gyrus (DG) electrical stimulation. The SFM was changed twice a week and the HOTCs were incubated at 36° C. with 5% CO₂-balance air. Slices were maintained to 21 DIV to allow cultures to mature, and used from 21-30 DIV. HOTC cultures remained stable from 21-30 DIV (i.e., showed no evidence of irreversible pyramidal neuron injury) using SFM or media with horse serum.

Spreading depression (SD) was used as a benign perturbation of HOTCs as a useful test of the normalcy of the HOTC. SD (without seizures) was induced in the HOTCs transitioned to SFM by simple repetitive electrical stimulation of the dentate gyrus, a stimulus analogous to that used in vivo. The HOTCs showed no injury from SD. However, when HOTCs were infused with serum-based media, seizures rather than SD resulted from the stimulation alone. Therefore, the transition to serum-free media after 4 DIV, which did not prompt a negative impact on glial or cytokine function, provided a marked improvement to HOTCs as an in vitro model for SD-induced neuroprotection.

### Example 12

### Activation of Spreading Depression in the Hippocampus

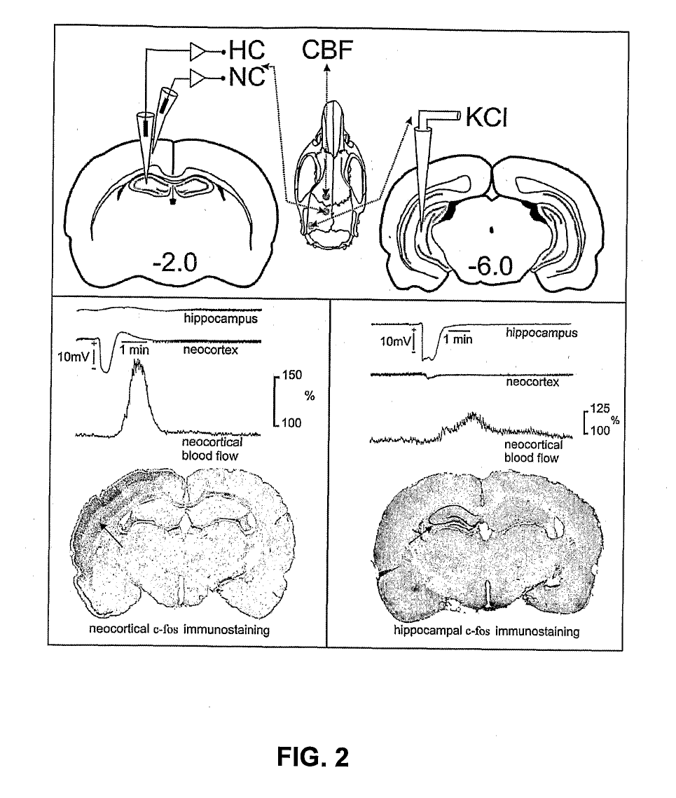

Regional brain activation from spreading depression was measured. The local reaction triggered by spreading depression is illustrated in FIG. 2 (neocortical spreading depression (lower left frame) and hippocampal spreading depression (lower right frame). The experimental method for triggering spreading depression is shown in FIG. 2, depicting a rat skull with gray circles indicating sites of craniotomies (FIG. 2, top center schematic). Direct current recordings were made through 1 of 2 craniotomies (−2.0 mm from bregma and 1.5 mm lateral to the midline) (FIG. 2, top left schematic). Two microelectrodes were glued together and were passed through this craniotomy so that the deeper microelectrode was 2800 μm below the pial surface in the hippocampus while at the same time the 2nd microelectrode was positioned 1000 μm into the neocortex. Spreading depression was induced by a microelectrode passed through the posterior craniotomy (−6.0 mm from bregma and 4.5 mm lateral to the midline) and lowered to a depth of 4500 μm below the pial surface for hippocampus spreading depression (FIG. 2, top right schematic) or 1000 μm below the pial surface for neocortex spreading depression using nL pulses of 1 M KCl.

A 3rd craniotomy was drilled over the frontal cortex (3 mm anterior to bregma and 2.0 mm lateral to the midline) to measure relative cerebral blood flow. A local reaction triggered by spreading depression is occurred in the neocortex and hippocampus. Neocortical interstitial direct current potential changes showed the typical negative direct current changes of spreading depression, triggered from nL injections of KCl. Neocortical spreading depression did not propagate to hippocampus but did trigger a local transient rise in blood flow during neocortical spreading depression to more than 150% of initial baseline level. Associated c-fos-immuno-staining changes following recurrent neocortical spreading depression were elicited every 9 minutes for 2 hours. Dense c-fos-immuno-staining was restricted to the ipsilateral neocortex which experienced spreading depression. Similarly, nL injections of KCl into the hippocampus triggered spreading depression there with associated local changes. Electrophysiological changes of spreading depression (FIG. 2, bottom schematic, top record) did not extend into the neocortex (FIG. 2, bottom schematic, middle record) yet electro-physiological changes did occur there. Hippocampal spreading depression induced a small negative-going shift in the neocortical direct current potential and reduced spontaneous neocortical electrical activity during and for minutes after spreading depression in hippocampus (FIG. 2, bottom schematic, bottom record). In addition, neocortical blood flow rose to 125% of control. The c-fos-immuno-staining immediately after recurrent hippocampal spreading depression was observed in the pyramidal cell layer and dentate gyrus of the ipsilateral hippocampus, but not in neocortex or contralateral hippocampus (FIG. 2, bottom schematic).

### Example 13

### Microglia Transfected with Coumarin-Loaded Nanoparticles Release the Nanoparticles to HOTC Cells

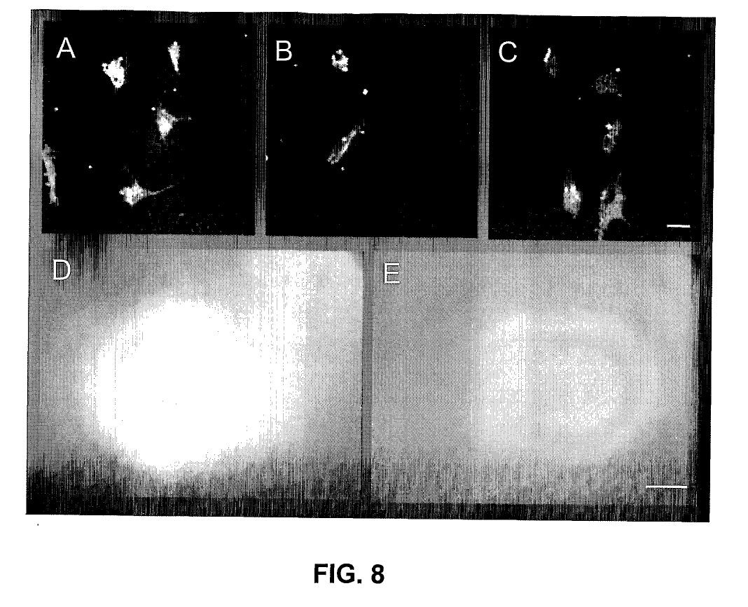

Microglia were loaded with 100 μg/mL coumarin-containing, 283 nm nanospheres for 1 hour and imaged using confocal fluorescence microscopy. Images were obtained via confocal microscopy and processed using Image J software and the “fire” LUT. The intensity of fluorescence of the microglia was measured at 0 hours, 4 hours and 18 hours after nanoparticle loading. Representative cells are shown in FIG. 8 A (0 hours), B (4 hours) and C (8 hours), and show that fluorescence intensity was less in the 4 hour sample than the 0 hour sample, and lower again in the 18 hour sample (i.e., coumarin nanospheres were lost from microglia over time). Calibration bar is 20 μm. Thus, PLGA nanospheres were released from the microglia.

Microglia in suspension were loaded with shaking at 37° C. with 640 μg/mL coumarin; cells were pelleted and resuspended twice with washing in serum free media (DMEM). 200 μL of the washed microglia were added to the top of HOTCs. The same concentration (640 μg/mL) of coumarin-loaded nanospheres was also directly applied to HOTCs. The intensity of fluorescence of the HOTC cells was examined using routine fluorescence microscopy in each instance 7 days later. Coumarin-loaded nanospheres contained in microglia marked HOTCs more intensely, causing much more fluorescence of the cells (FIG. 8, D) than HOTCs exposed to coumarin-loaded nanospheres alone (FIG. 8, E).

### Example 14

### Enhancement of Movement of Coumarin-Loaded Nanoparticles from Microglia into HOTC Tissue by Neural Activity

### Spreading Depression

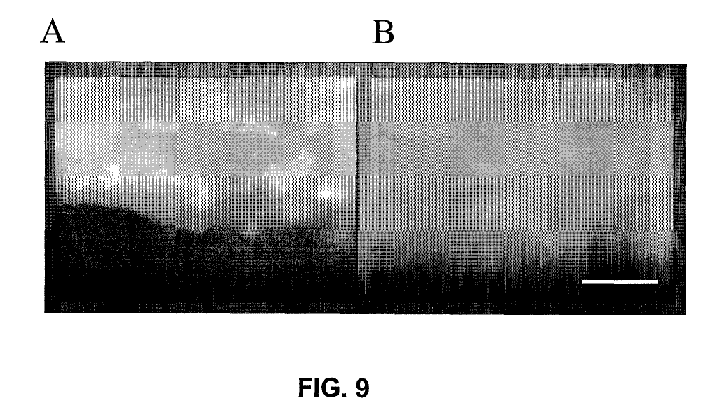

Spreading depression (SD) was elicited using a typical single bipolar electrical stimulus to the dentate gyrus. SD was confirmed by a mono polar DC recording electrode placed in the CA3 area of HOTC. SD was evoked every 9 min for one hour, 100 μL of a 1:1× solution of suspended microglia previously loaded with 200 μg/mL nanospheres via shaking for 15-30 minutes at 37° C. was added to the top of an HOTC that experienced SD (FIG. 9, A) and a control, normal HOTC (FIG. 9, B). The microglia more quickly traveled into an HOTC that experienced SD (FIG. 9, A) compared to the control culture (FIG. 9, B). Five days later HOTCs were fixed with fixative and examined for evidence of microglia penetration into HOTCs using routine fluorescence microscopy. No fluorescence above background auto fluorescence was seen in any normal HOTCs (FIG. 9, B) or in any HOTC on the same insert as the single one that experienced SD. On the other hand, coumarin (green) fluorescence was seen penetrating at least 500 μm into the HOTC that experienced SD (FIG. 9, A).

### Example 15

### Prophetic

### Determination of the Optimal Nanoparticle Size for Expression of Polynucleotides in Macrophages, Microglia and Other Brain Cells (in HOTCs) Via Polynucleotide Carrying Nanospheres Loaded into Macrophages

Bone marrow derived macrophages and microglia are plated at a uniform density (˜100,000 cells/mL) and then exposed to nanospheres (50, 100, 150, 200 and 250 nm in diameter) made from PLGA (or mPEG-PLGA) as sham controls (i.e., without DNA but with coumarin) at concentrations of 20, 40, 80, 160, 320, and 640 μg/mL nanospheres for 1 hour, 3 hours or continuously. Half of the culture media is changed daily with astrocyte-conditioned media (harvested daily from confluent astrocyte cultures grown in 75 cm² flasks).

Cells are harvested for analyses 1, 3, 7, 14, 21, and 28 days later. Analyses include efficiency of nanosphere uptake into cells. Uptake efficiency is determined using high performance liquid chromatography, fluorescence microscopy (or confocal microscopy), and for the fluorescent marker coumarin and electron microscopy for nanospheres. Parallel analyses are completed for toxicity (measured with fluorescent markers (e.g., from Invitrogen), for apoptosis (via routine fluorescence microscopy) and inflammatory reaction, for pro- and anti-inflammatory cytokines, (via multiplexed proteomic ELISA assays). Cell density measurements are used to assess proliferation of the cells.

Other cells (with nanospheres that include the plasmid for GFP (1 μg DNA/3 mg PLGA (143 kD MW, 50:50 lactide:glycolide ratio) or mPEG-PLGA) are treated as above and analyzed for GFP protein expression (using routine fluorescence microscopy and confocal fluorescence microscopy for direct confirmation and bright field microscopy (for GFP immunostaining) and electron microscopy (using immuno gold staining) for indirect, but more sensitive, confirmation. Finally, real-time PCR analyses for GFP mRNA are used as a most sensitive confirmation of GFP plasmid expression in macrophages/microglia.

Microglia are used as a positive control for macrophages in both classes of experiment above.

### Example 16

### Prophetic

### Optimization of Polynucleotide Expression in Macrophages and Microglia and in Other Neural Cells Via Delivery of Macrophages Containing the Polynucleotides in Nanoparticles Under Normal Conditions and Excitotoxic Injury

Bone marrow derived macrophages and microglia are plated at a uniform density (˜100,000 cells/mL) and then exposed to nanospheres (50, 100, 150, 200 and 250 nm in diameter) made from PLGA and mPEG-PLGA as sham controls (i.e., without DNA but with coumarin) at concentrations of 20, 40, 80, 160, 320, and 640 μg/mL nanospheres for 1 hour, 3 hours or continuously. Half of the culture media is changed daily with astrocyte-conditioned media (harvested daily from confluent astrocyte cultures grown in 75 cm² flasks).

Macrophage and microglial cells are harvested for transplantation to HOTCs after 1, 3 or 8 days for tests involving reservoir delivery. This is done by scraping cells free from glass coverslips or by brief expose of cells to trypsin which is then quenched by addition of normal growth media. Cells are pelleted via centrifugation, resuspended, and added (100-200 μL) to the insert area above HOTCs.

For cell-specific delivery to neurons, astrocytes, and oligodendrocytes bone-marrow derived macrophages grown as described above are more briefly loaded (for 5, 15 or 30 minutes) with coumarin-based nanospheres (or equivalent nanospheres containing the plasmid DNA encoding GFP) to promote recycling exocytosis when cells are exposed to brain tissue. To initially retain nanospheres within cells, protein from surrounding media is removed by washing twice with DMEM (via centrifugation, pelleting, and resuspension). With the third resuspension in DMEM, cells are added (100-200 μL) to the insert area above HOTCs. HOTCs are treated with macrophage containing nanospheres after 21 days in vitro (DIV) and sampled for experimental endpoints 1, 3, 7, 14 and 21 days after transplantation.

Endpoint analyses are performed as described in Example 14. Efficiency of nanosphere uptake is determined using high performance liquid chromatography, fluorescence microscopy (or confocal microscopy), and for the fluorescent marker coumarin and electron microscopy for nanospheres. Parallel analyses are completed for toxicity (measured with fluorescent markers (e.g., from Invitrogen), for apoptosis (via routine fluorescence microscopy) and inflammatory reaction, for pro- and anti-inflammatory cytokines, (via multiplexed proteomic ELISA assays (see [25], herein incorporated by reference).

Other cells (containing nanospheres that include the plasmid for GFP (1 μg DNA/3 mg PLGA (143 kD MW, 50:50 lactide:glycolide ratio) or mPEG-PLGA) are treated as above. These cells are analyzed for GFP protein expression using routine fluorescence microscopy and confocal fluorescence microscopy for direct confirmation and bright field microscopy for GFP immunostaining. Electron microscopy (using immuno gold staining) is used as indirect, but more sensitive, confirmation of expression. Real-time PCR analyses for GFP mRNA are used as a most sensitive confirmation of GFP plasmid expression in HOTC tissue. Cell-specific expression of GFP protein is determined using confocal microscopy plus cell-specific immunostaining and immuno gold staining coupled to electron microscopy. Measurements of cell-specific transfection efficiency are made by determining the number of GFP positive cells compared to non positive cells (per cell type) for areas of interest.

HOTCs (21 DIV) are injured by 1 hour exposure to oxygen glucose deprivation to trigger selective neuronal loss of CA1 area pyramidal neurons. This is accomplished by moving HOTCs to a serum free media without glucose and exposing them to <1% oxygen atmosphere (at 10% carbon dioxide-remainder nitrogen) at 37° C. Afterwards, HOTCs are returned to normal media and incubation until they are harvested as above at 1, 3, 7, 14 and 21 days after injury. Endpoints are to determine the extent and distribution of GFP expression within macrophages and microglia as well as expression of GFP in neurons, astrocytes and oligodendrocytes. Tissue injury (using fluorescent markers of cell death coupled to fluorescence microscopy) and tissue inflammation (using measurements of pro- and anti-inflammatory cytokine production) responses are determined.

In other experiments, cell-specific expression of GFP is enhanced by using polynucleotide-carrying nanospheres where the plasmid for GFP is altered to carry a cell-specific promoter. To achieve astrocyte-restricted expression, GFP is placed under control of the human GFAP promoter (GenBank Accession #M67446). The portion of the human GFAP that directs astrocyte-specific expression is amplified from human genomic DNA using PCR methods

To direct neuron-specific reporter gene expression, GFP is placed under the control of the rat neuron-specific enolase (NSE) promoter (GenBank Accession #ABO38993). The rat NSE promoter directs efficient and specific expression in neurons and has been used to drive GFP expression in neurons both in primary culture and in vivo.

To achieve oliogodendrocyte specific reporter gene expression of GFP, GFP expression is placed under the control of myelin basic protein (MBP; or other oligodendrocyte-specific) promoter. Resulting clones are sequenced to verify sequence integrity. This is to ensure that while other cells may take up nanospheres carrying a GFP plasmid, only those operative for the cell specific promoter will express the GFP protein.

Microglia are used as a positive control for macrophages in both classes of experiment above.

### Example 17

### Prophetic

### Delivery of Reservoir Macrophages and Microglia to Normal and Injured Brain in Whole Animals, and Cell-Specific Expression of Plasmid DNA Delivered to Brain via Nanoparticles Comprising Plasmid DNA Contained in Macrophages

Bone marrow derived macrophages are plated at a uniform density (˜100,000 cells/mL) and then exposed to nanospheres (50, 100, 150, 200 and 250 nm in diameter) made from PLGA (or mPEG-PLGA) as sham controls (i.e., without DNA but with coumarin) at concentrations of 20, 40, 80, 160, 320, and 640 μg/mL nanospheres for 1 hour, 3 hours or continuously. Half of the culture media is changed daily with astrocyte-conditioned media (harvested daily from confluent astrocyte cultures grown in 75 cm² flasks).

Cells are harvested for transplantation to animals after 1, 3 or 8 days for tests involving reservoir delivery. This is done by scraping cells free from glass coverslips or by brief expose to trypsin which is then quenched by addition of normal growth media. Cells are pelleted via centrifugation and resuspended.

For cell-specific delivery (to neurons, astrocytes, and oligodendrocytes) bone-marrow derived macrophages grown as described above are more briefly loaded (5, 15 or 30 minutes) while in suspension with coumarin-based nanospheres (or equivalent nanospheres containing the plasmid with DNA encoding GFP) to promote recycling exocytosis when cells are exposed to brain tissue. To initially retain nanospheres within cells, protein from surrounding media is removed by washing twice with DMEM via centrifugation pelleting and resuspension. With the third resuspension in DMEM, cells are added (100-200 μL) to the insert area above HOTCs. HOTCs are treated with macrophage containing nanospheres after 21 days in vitro (DIV) and sampled for experimental endpoints 1, 3, 7, 14 and 21 days after transplantation.

Cell-specific expression of GFP is enhanced by using polynucleotide-carrying nanospheres where the plasmid for GFP is altered to carry a cell-specific promoter. This ensures that while other cells may take up nanospheres carrying a GFP plasmid, only those capable of transcribing the cell specific promoter will express the GFP protein.

Three strategies are used for delivery of macrophages to brain. Macrophages are loaded (reservoir delivery) in primary culture or suspension (bystander delivery) and harvested for delivery into whole animals as described in Example 16 with the following additions. Delivery to brain is accomplished via sterile intra-arterial injection, intra-thecal injection, or nasal mucosa administration to anesthetized (50 mg/kg intra peritoneal injection of pentobarbital with adequate anesthesia determined by absence of withdrawal to paw pinch) rats. Dose-response injection studies involve using 4-6×10⁶ cells, then 10⁵ cells, and finally 10⁷ cells in 100-400 μL of DMEM over 1-5 minutes. Arterial injections use the left vertebral artery and intra-thecal injections are done at the atlanto-occipital membrane. Both are accomplished via sterile surgical site at the left dorsal neck. After injections, the surgical site is coated with bupivacaine and closed with sutures. Intra-nasal injections are accomplished with the anesthetized rat in a prone position and cells applied to the nasal mucosa via a fine polyethylene catheter.

Experimental endpoints are to determine the efficiency of GFP expressing macrophage/microglia transplantation to brain, the duration of expression, cell specificity of expression, and any other organ confounding expression as well as neural toxicity and tissue inflammation from transplantation from each route of delivery and dose per route. Accordingly, after injections animals are allowed to survive for 1, 3, 7, 14, and 21 days before they are re-anesthetized and killed by perfusion fixation and harvesting of brain tissue for histological analyses, decapitation and harvesting of brain for inflammatory mediator analyses. Contralateral brain, lung and liver are examined for non targeted uptake of GFP plasmid containing nanospheres assessed via real-time PCR.

Stereological histological procedures are used for bright-field immuno-staining analysis of GFP expression in macrophages or other neural cells. Similar analyses of cell-specific expression of GFP are done using cell-specific immunostaining and confocal microscopy as well as immuno gold staining for GFP using electron microscopy. Toxicity is measured using FluoroJade B staining (which marks dying neurons) and compromised spatial memory. Inflammation is determined using analyses of pro- and anti-inflammatory cytokine expression. In each case, the density, morphology, and cell specificity of GFP expression is determined over time.

The above transfection and transplantation procedures are duplicated after CA3 neuronal excitotoxic injury to assess the potential cellular responses available for treatment after an exemplary model of excitotoxic injury. Here, awake rats are given an intra-peritoneal injection of kainic acid (10 mg/kg) to simulate death of CA3 neurons from temporal lobe status epilepticus after association with temporal lone seizures. Then, 0, 1, and 3 hours later cell deliveries (reservoir macrophages and bystander loading macrophages) are administered (via arterial injection, intra thecal injection or nasal administration). Animals are observed for 5 hours after kainic acid injections. Should continuous seizures occur for more than 5 minutes, animals are immediately euthanized. Otherwise, animals are returned to individual cages and observed at least daily until they again are re-anesthetized for brain harvests and endpoint measurements as described above 1, 3, 7, 14 and 21 days later.

These animals serve as sham control delivery counterparts for the excitotoxic injury animals of Examples 18 and 19.

### Example 18

### Prophetic

### Amelioration of Signs and Symptoms of Temporal Lobe Epilepsy (Excitotoxic Injury and Learning and Memory Effects) by Reservoir Expression of Cu/Zn-Superoxide Dismutase in Transplanted Macrophages

Bone marrow derived macrophages are plated at a uniform density (˜100,000 cells/mL) and then exposed to nanospheres (50, 100, 150, 200 and 250 nm in diameter) made from PLGA (or mPEG-PLGA) and containing the plasmid for GFP (as a sham control (see Example 16)) or the plasmid comprising a polynucleotide encoding Cu/Zn-Superoxide dismutase (Cu/Zn-SOD) at concentrations of 20, 40, 80, 160, 320, and 640 μg/mL nanospheres for 1 hour, 3 hours or continuously. Half of the culture media is changed daily with astrocyte-conditioned media (harvested daily from confluent astrocyte cultures grown in 75 cm² flasks).

Cells are harvested for transplantation to animals after 1, 3 or 8 days. This is done by scraping cells free from glass coverslips or by brief expose to trypsin which is then quenched by addition of normal growth media. Cells are pelleted via centrifugation and resuspended.

Exemplary neurodegeneration from excitoxic injury, simulating temporal lobe epilepsy, is triggered in awake rats via an intra peritoneal injection of kainic acid (10 mg/kg). Then, 0, 1, and 3 hours later cell deliveries of Cu/Zn-SOD expressing macrophages are administered via vertebral artery injection, intra-thecal injection, or intra-nasal application as described in Example 17. Animals are observed for 5 hours after kainic acid injections. Should continuous seizures occur for more than 5 minutes, animals are immediately euthanized. Otherwise, animals are returned to individual cages and observed at least daily until they again are re-anesthetized for brain harvests 1, 3, 7, 14 and 21 days after treatment.

Endpoints are to determine the degree (magnitude, spatial extent, and duration) of Cu/Zn-SOD expression in macrophages (via immunostaining) and the degree to which treatment with Cu/Zn-SOD expressing macrophages lessens the severity of kainic acid injury (as measured by FluoroJade B staining of hippocampus and altered spatial memory which involves caudal hippocampus). In addition tissue inflammatory responses are measured via multiplexed ELISAs for pro- and anti-inflammatory cytokines 1, 3, 7, 14 and 21 days after injury.

Increased reservoir expression of Cu/Zn-SOD in microglia is expected to reduce selective neuronal loss in CA3 (plus associated tissue inflammation) compared to sham controls (i.e., animals treated with GFP expressing macrophages (see Example 16)) since microglia can contribute to excitotoxic injury when activated to a phagocytic level by free radicals. Reduced activation by increased free radical scavenging from elevated exogenous expression of Cu/Zn SOD is expected to reduce pathological activation of these cells. Furthermore, increased expression of Cu/Zn-SOD in neurons is expected to reduce spatial learning, whereas mild activation of microglia promotes learning. Thus, increased Cu/Zn SOD expression in microglia is expected to increase rather than decrease hippocampal learning. Accordingly, measurements of spatial learning are done 7, 14 and 21 days after treatment and compared to normal and sham (i.e., GFP expressing macrophages) treated animals.

### Example 19

### Prophetic

### Amelioration of Temporal Lobe Epilepsy Symptoms (Induced Excitotoxic Injury by Kainic Acid) without Altering Learning and Memory by Cell-Specific Expression of the Polynucleotide Encoding Cu/Zn-SOD Contained in Nanospheres Delivered to Brain in Macrophages

For cell-specific delivery (to neurons, astrocytes, and oligodendrocytes) bone-marrow derived macrophages grown as described in Example 15 are briefly loaded (5, 15 or 30 minutes) with nanospheres carrying plasmid DNA for Cu/Zn-SOD to promote recycling exocytosis when cells are exposed to brain tissue. To initially retain nanospheres within cells, protein from surrounding media is removed by washing twice with DMEM via centrifugation, pelleting, and resuspension. With the third resuspension in DMEM, cells are loaded into a syringe and delivered to brain as described in Example 17 after kainic acid exposure to induce epilepsy symptoms (also as described in Example 17).

Endpoints include the determination of the degree (magnitude, spatial extent, and duration) of Cu/Zn-SOD expression in macrophages (via immunostaining) and the degree to which treatment with Cu/Zn-SOD expressing macrophages lessens the severity of kainic acid injury (as measured by FluoroJade B staining of hippocampus and altered spatial memory which involves caudal hippocampus). In addition tissue inflammatory responses are measured via multiplexed ELISAs for pro- and anti-inflammatory cytokines 1, 3, 7, 14 and 21 days after injury. The extent, magnitude, and duration of cell-specific expression of Cu/Zn SOD will be determined using immunostaining for Cu/Zn SOD and cell-specific immunostaining with results viewed via confocal microscopy.

Increased neuronal expression of Cu/Zn-SOD is expected to have the greatest impact on ameliorating excitotoxic injury and also the greatest impact on inhibiting post treatment learning and memory, while expression in astrocytes and microglia will have a lesser impact. Expression of Cu/Zn-SOD in oliogodendrocytes is expected to reduce their loss from excitotoxic injury, and help ameliorate neonatal stroke and hypoxic/ischemic injury.

### Example 20

### Prophetic

### Impact of Increased Neural Activity on Reservoir and Cell-Specific Expression of Cu/Zn-SOD as a Therapeutic to Lessen Temporal Lobe Epilepsy Symptoms (Induced by Excitotoxic Injury with Kainic Acid) and Associated Potential Inhibition of Learning and Memory

Hippocampal neuronal activity is increased naturally by exposing animals, e.g., rats, to an enriched environment, such as providing group-housed animals with weekly changes of novel tunnels for exploration. This results in an enhancement of hippocampal learning and memory, and thus electrical activity in the hippocampus. Such increased neural activity occurs with low-level increased pro-inflammatory cytokine expression that may enhance macrophage chemotaxis. Accordingly, the experiments detailed in Examples 17 and 18 are repeated using the animals that have been exposed to an enriched environment for 28 days.

In a second aspect of this experiment, neuronal activity is increased by theta burst bipolar electrical stimulation in anesthetized rats (50 mg/kg intra peritoneal pentobarbital) given every 5 min for 30 minutes. This is followed (0, 1 and 3 hours later) by reservoir or cell-specific delivery of increased Cu/Zn SOD expression as described in Examples 17 and 18, respectively. Sham controls consist of analogous injections but without electrical stimulation.

Endpoints determine the degree (magnitude, spatial extent, and duration) of Cu/Zn-SOD expression in macrophages (via immunostaining) and for cell-specific expression of Cu/Zn-SOD using immunostaining for Cu/Zn-SOD and cell-specific immunostaining with results viewed via confocal microscopy.

Increased neural activity is expected to amplify the impact of cell-specific increased treatment expression of Cu/Zn SOD described in Example 19.

### Example 21

### Prophetic

### Tumor Cell-Specific Delivery of Nanoparticles Carried in Macrophages and Containing RNAi for Epidermal Growth Factor Receptor (EGFR) to Reduce Disease Severity from Primary Glioma

Intra-cerebral injections of rat glioma cells (i.e., F98 or RG2 (Catalog #CRL-2397 and catalog #CRL-2433, respectively from American Type Culture Collection) are made in anesthetized (50 mg/kg intra peritoneal pentobarbital) rats (adult male CD Fisher 344). Anesthetized rats are placed in a standard stereotaxic unit and a sterile, 1 cm incision is made over left frontal cortex. The skin is retracted and underlying bone scraped clean. A 2 mm burr hole is placed 2 mm lateral and 2.5 mm anterior to bregma without tearing the underlying dura. Then, glioma cells (5×10⁴ cells in 2 μL) are injected via a 10 μL Hamilton syringe attached to a 26 gauge needle inserted 3,500 μm below the cortical surface. Injection rate is over 5 minutes. The injection needle is left in place for another 5 minutes before withdrawal. The craniotomy site is closed with bone wax and the incision site infiltrated with bupivacaine before being closed with sutures. Animals are allowed to recover from anesthesia before being returned to individual cages under standard housing conditions. They are observed daily until harvest.

EGFR RNAi plasmid is constructed using standard, published protocols. Cell-specific delivery to tumors is enhanced by incorporating the glioma-selective promoter, nestin, into plasmids. Plasmid DNA is incorporated into PLGA (or mPEG-PLGA nanospheres) and loaded into macrophages as described in Examples 15 and 16.

For cell-specific delivery (to neurons, astrocytes, and oligodendrocytes) macrophages are more briefly loaded (5, 15 or 30 minutes) while in suspension with nanospheres loaded with EGFR RNAi plasmid. To initially retain nanospheres within cells, protein from surrounding media is removed by washing twice with DMEM via centrifugation pelleting and resuspension.

Delivery to brain is accomplished via sterile intra carotid injections to the left common carotid artery to guide nanosphere carrying macrophages to left brain hemisphere of anesthetized (50 mg/kg intra peritoneal injection of pentobarbital with adequate anesthesia determined by absence of withdrawal to paw pinch) rats. After injections, rats are allowed to recover in individual cages before being re-anesthetized 7 or 14 days after treatment and killed by perfusion fixation, decapitated and harvesting of brain. Injection of macrophages carrying the GFP plasmid (with the nestin promoter) serves as the sham control.

Experimental endpoints are to determine tumor size (via stereological analyses of routine staining of serial histological sections), EGFR expression (via hemispheric western blot analyses, and immunostaining (examined using routine fluorescence or bright field microscopy) versus sham controls. In addition, non specific delivery of EGFR shRNA nanosphere carrying macrophages to the right brain hemisphere, lung and liver will be determined using real-time PCR for the shRNA.

Tumor cell specific suppression of EGFR expression is expected to reduce the clinical impact of primary brain tumor modeled in rat by prolonging survival, reducing tumor burden, and reducing astrogliosis from brain tumor injury.

### Example 22

### Prophetic

### Mitigation of Temporal Lobe Epilepsy by Delivery of Macrophages Carrying Nanospheres Loaded with a Plasmid to Enhance Cellular Release of Galanin

A well-established animal model of temporal lobe epilepsy (TLE) is used to deliver nanosphere-macrophage-based DNA therapy to provide a long-lasting reservoir of an endogenous neuropeptide with anti-convulsant effects. TLE is induced in adult rats by an intra-peritoneal injection of kainic acid (10 mg/kg (Sigma) in PBS (at 5 mg/mL).

Seizure severity is quantitated for the next 5 hours according to a scale known in the art proposed by Racine, and modified to include death: 0=normal behavior; 1=immobility; 2=forelimb and/or tail extension, rigid posture; 3=repetitive movements, head bobbing; 4=rearing and falling; 5=continuous rearing and falling; 6=tonic-clonic seizures; 7=death. Animals are monitored continuously for 5 hours and given a score every 20 minutes that represented the highest seizure severity for that period. The maximal score each hour is used to indicate seizure severity. In the event that animals experience continuous seizures (i.e., levels 5 or 6) for 10 minutes, animals are immediately euthanized.

The galanin plasmid is constructed according to T J McCown (Adeno-associated virus-mediated expression and constitutive secretion of galanin suppresses limbic seizure activity in vivo, Mol Ther 14:63-68, 2006) and example 1 and enclosed into nanoparticles as described in example 2 and then transfected ex vivo macrophages grown in culture as described in example 9. Plasmid injections are performed (as described in example 17) 1 day before and 1, 3, and 7 days after kainic acid injections except that arterial injections are completed via both vertebral arteries.

Experimental endpoints are the latency to class 1, 2, 3, 4, 5, 6, and 7 limbic seizure behavior using observation periods of 20 minutes a day every day after seizure induction; degree of CA3 pyramidal neuron loss measured by Fluoro Jade B staining (as described in example 17). Sham control is injection of macrophages carrying nanospheres with the GFP plasmid. Non specific expression of galanin is measured in contralateral hemisphere, lung and liver via real-time PCR.

Galanin is an endogenous neuropeptide with anti-seizure activity. Accordingly, increased microglial expression of galanin is expected to reduce seizure severity and associated CA3 pyramidal neuron loss from kainic acid-induced limbic seizures.

### Example 23

### Prophetic

### Lessening of Chronic Pain by Delivery of Macrophages Carrying Nanospheres Loaded with a Plasmid to Enhance Cellular Release of interleukin-10 (IL-10) and Augmentation of this Process by Peripheral Nerve Stimulation.

Chronic constriction injury (CCl) is created at the mid-thigh level of the left hind leg of a rat by tying a resorbable 4-0 chromic gut suture loosely around the isolated sciatic nerve under sterile conditions and pentobarbital (50 mg/kg intra-peritoneal injection) anesthesia. Sham operated rats are surgically manipulated but no sutures are tied around the sciatic nerve. Wounds are closed with suture material and animals are allowed to recover in individual cages. Three and 10 days later, animals are tested for allodynia with the Von Frey test and the threshold for behavioral response to heat stimuli. An upper limit to the latter is set at 20 seconds to avoid tissue damage.

The IL-10 plasmid is constructed according to ED Milligan et al. Controlling pathological pain by adenovirally driven spinal production of the of the anti-inflammatory cytokine, IL-10 Eur J Neurosci 21:2136-2148, 2005, incorporated herein by reference in its entirety, and enclosed into nanoparticles as described in Example 2. The nanoparticles are transfected ex vivo into macrophages grown in culture as described in Example 9. Plasmid injections are performed as described in Example 17. Measurements are made 3 and 10 days after treatments. After pain measurements at each of these times, animals are re-anesthetized and killed by decapitation. The spinal cords are removed and the sacral area of the cords assessed for IL-10 expression using an ELISA.

Endpoints are to establish the degree to which increased IL-10 from transplanted macrophages reduces pain and increases IL-10 expression in spinal cord. We expect that macrophage expressing IL-10 treatment will reduce evidence of chronic pain.

### Example 24

### Prophetic

### Reducing Impact of Experimental Multiple Sclerosis by Delivery of Macrophages Carrying Nanospheres Loaded with a Plasmid to Enhance Cellular Release of FGF-2

To trigger the induction of experimental allergic encephalitis (EAE), adult Lewis rats are immunized with myelin basic protein (MBP) in complete Freund's adjuvant according to standard protocols. Animals are observed daily and given a clinical score that is the composite of the degree of paresis of each limb and tail (partial paresis, 0.5; complete paresis, 1.0). Animals that reach a score of 5 are immediately withdrawn from study and euthanized. Otherwise, animals are maintained in individual cages until harvest at 1, 2, 4, and 6 weeks later by decapitation or trans-cardiac perfusion with fixative under intra-peritoneal pentobarbital (50 mg/kg).

The FGF-2 plasmid is constructed according to F Ruffini et al., Fibroblast growth factor-II gene therapy reverts the clinical course and the pathological signs of chronic experimental autoimmune encephalomyelitis in C57BL/6 mice, Gene Ther 8:1207-1213, herein incorporated by reference in its entirety. The FGF-2 plasmid is enclosed into nanoparticles as described in Example 2 and transfected into ex vivo macrophages grown in culture as described in Example 9.

Experimental endpoints are to determine the degree of clinical improvement with treatment versus the sham of GFP plasmid expression. Similar comparisons are done for expression of FGF-2 (measured via ELISA from cortical samples), T-cell and macrophage invasion of cortical tissue (measured via cell-specific immunostaining), and the number of myelin precursors and myelin-forming oligodendrocytes (measured via immunostaining).

The use of macrophages to increase expression of FGF via nanospheres carrying the FGF plasmid is expected to provide protective effects against experimental allergic encephalitis and multiple sclerosis.

All patents, publications and references cited herein are hereby fully incorporated by reference. In cases of conflict between the present disclosure and incorporated patents, publications and references, the present disclosure should control.

What is claimed is:

1. A method of delivering a therapeutic agent to the brain of a mammal comprising:
(a) contacting a bone marrow stem cell, monocyte, macrophage, or microglia ex vivo with a nanoparticle comprising the therapeutic agent under conditions that allow endocytosis of the nanoparticle by the cell;
(b) delivering the cell of step (a) to the brain.

2. The method of claim 1, wherein after step (a) and prior to step (b) the cell is maintained under conditions that maintain the nanoparticle inside the bone marrow stem cell, monocyte, macrophage, or microglia.

3. The method of claim 1, wherein the cell is delivered to the brain by regional intra-arterial delivery, intra-thecal delivery, intra-peritoneum delivery, intra-venous delivery or intra-nasal delivery.

4. The method of claim 1, wherein the therapeutic agent is encapsulated within the nanoparticle.

5. The method of claim 1, wherein the nanoparticle comprises poly(lactic-co-glycolic acid).

6. The method of claim 5, wherein the nanoparticle further comprises methoxypolyethylene glycol.

7. The method of claim 1, wherein the nanoparticle further comprises avidin, biotin or a combination thereof.

8. The method of claim 1, wherein the nanoparticle further comprises a ligand specific for a receptor or cell surface marker on the cell of step (a) or on a brain cell.

9. The method of claim 8, wherein the ligand is an antibody.

10. The method of claim 1, wherein the diameter of the nanoparticle is less than about 500 nm.

11. The method of claim 1, wherein the diameter of the nanoparticle is less than about 250 nm.

12. The method of claim 1, wherein the diameter of the nanoparticle is from about 50 to about 100 nm.

13. The method of claim 1, wherein the therapeutic agent is a polynucleotide, a polypeptide, or a small molecule.

14. The method of claim 13, wherein the therapeutic agent comprises a polynucleotide coding sequence encoding a polypeptide, an RNAi-inducing agent, or an antisense RNA, the coding sequence operably linked to a promoter.

15. The method of claim 14, wherein the coding sequence is expressed in the bone marrow stem cell, monocyte, macrophage or microglia.

16. The method of claim 13, wherein the therapeutic agent is a polynucleotide comprising or encoding an RNAi-inducing agent or an antisense RNA.

17. The method of claim 14, wherein the promoter is a cell-specific promoter.

18. The method of claim 17, wherein the promoter is an astrocyte-specific promoter, an oligodendrocyte-specific promoter, a neuron-specific promoter, a microglia-specific promoter, or an endothelial cell-specific promoter.

19. The method of claim 1, wherein the therapeutic agent is a chemotherapeutic agent, an anti-angiogenic agent, a cytotoxic agent, a cytokine, a growth factor, an apoptotic pathway effector, neurotransmitter precursor, a neurotransmitter agonist or antagonist, an antibody, a radionuclide, an reactive oxygen species scavenger, an anti-inflammatory agent, an analgesic, an antagomir, a radioactive agent, or an imaging agent.

20. The method of claim 13, wherein following step (b), the therapeutic agent contacts or is taken up by a brain cell.

21. The method of claim 20, wherein the therapeutic agent is a polynucleotide and wherein the polynucleotide is taken up by and expressed in a brain cell.

22. The method of claim 1, further comprising enhancing delivery to a region of the brain by magnetic or stimulant treatment.

23. A method of producing a bone marrow stem cell, monocyte, macrophage or microglial cell for delivering a therapeutic agent to a tissue comprising:
(a) contacting the cell ex vivo with a nanoparticle comprising the therapeutic agent comprising a polynucleotide under conditions that allow endocytosis of the nanoparticle by the cell.

24. The method of claim 23, further comprising following step (a) and prior to delivering the therapeutic agent, maintaining the cell under conditions that maintain the nanoparticle inside the bone marrow stem cell, monocyte, macrophage, or microglia.

25. The method of claim 24, wherein the nanoparticle is maintained nanoparticle inside the bone marrow stem cell, monocyte, macrophage, or microglia by a reduction in exocytosis.

26. The method of claim 25, wherein the conditions comprise maintaining the cell in a medium substantially free of serum or protein.

27. The method of claim 23, wherein the polynucleotide encodes a polypeptide, an RNAi-inducing agent or an antisense RNA.

28. A composition for delivering a therapeutic agent to a tissue, comprising a bone marrow stem cell, monocyte, macrophage or microglial cell comprising a nanoparticle comprising the therapeutic agent, and a medium substantially free of serum or protein.

29. The composition of claim 28, wherein the nanoparticle comprises a synthetic polymer.

30. A composition for delivering a therapeutic agent to a tissue, the composition comprising a bone marrow stem cell, monocyte, macrophage or microglial cell comprising a nanoparticle comprising the therapeutic agent, wherein the therapeutic agent comprises a polynucleotide.

31. The method of claim 30, wherein the nanoparticle comprises a synthetic polymer.
